(19) United States
(12) Patent Application Publication
(10) Pub. No.: US 2008/0312557 A1
(43) Pub. Date: Dec. 18, 2008

(54) Method and system for measuring urinary flow rate

(76) Inventors: Dong Uk Cho, Goesan-Gun (KR); Kyung Ah Kim, Cheongju-City (KR); Eun Jong Cha, Cheongju-City (KR)

Correspondence Address: GREER, BURNS & CRAIN, 300 S WACKER DR, 25TH FLOOR, CHICAGO, IL, 60606, US

(21) Appl. No.: 11/820,086

(22) Filed: Jun. 18, 2007

(51) Int. Cl. A61B 5/20

(52) U.S. Cl. 600/584; 604/322

(57) ABSTRACT

Disclosed are a method and an apparatus capable of efficiently and precisely measuring a urinary flow rate by using a general purpose pressure sensor with a low price, instead of a conventional expensive weight sensor, even without a conventional funnel. According to the method and the apparatus, water pressure of urine accumulated in a vessel is transferred to an external pressure sensor through a pressure transfer pipe, the received water pressure is converted into electrical signals through the pressure sensor, the electrical signals are amplified through a bridge amplifier, the amplified analog signals into digital signals (water pressure signals), the water pressure signals are converted into volume signals through an interface with an analyzer, and urinary flow rate signals are acquired by differentiating the volume signals, in which the electrical signals are voltage signals corresponding to volume signals.

An apparatus capable of measuring a urinary flow rate. According to the method and the apparatus, water pressure of urine accumulated in a vessel is transferred to an external pressure sensor through a pressure transfer pipe, the received water pressure is converted into electrical signals through the pressure sensor, the electrical signals are amplified through a bridge amplifier, the amplified analog signals into digital signals (water pressure signals), the water pressure signals are converted into volume signals through an interface with an analyzer, and urinary flow rate signals are acquired by differentiating the volume signals, in which the electrical signals are voltage signals corresponding to volume signals.

### BACKGROUND OF THE INVENTION

1. Field of the Invention

The present invention relates to a method and an apparatus for measuring a urinary flow rate. More particularly, the present invention relates to a method and an apparatus for measuring a urinary flow rate using a manometer.

2. Description of the Related Art

In general, the masculine urinates through the urethra surrounded by the prostate under the bladder. The prostate is a line-shaped organ exclusively owned by the masculine. The prostate surrounds the urethra under the bladder and is a soft tissue having a chestnut shape with a weight of about 20 g. A prostatomegaly denotes a disease that the prostate is hypertrophied due to inflammation. The prostatomegaly is a representative chronic disease caught by the masculine in the aging society. In particular, about 50 to 80% of the masculine having ages of fifties or more suffers from the prostatomegaly. If the prostatomegaly is broken out, the urethra surrounded by the prostate is compressed, and thus sexual dysfunction appears as well as_urination disorder.

In order to determine the disease in the prostate, i.e. the prostatomegaly, an uro-flowmetry scheme is used to continuously measure urinary flow rate signals indicating the amount of urine per unit time during the urination. This scheme is an essential living body measurement inspection required for diagnosing the prostatomegaly.

The conventional uro-flowmetry scheme is based on the measurement of the weight of urine. According to the uro-flowmetry scheme as schematically shown in FIG. 1, urine 13 is accumulated in a vessel 10 having a constant diameter, and a weight sensor 12 attached to the bottom of the vessel 10 measures the change in weight of the urine during the urination, in which the weight sensor 12 is a load-cell device measuring weight. This method uses the fact that weight measured by the weight sensor 12 is proportional to the volume of the urine contained in the vessel 10. That is, when the weight measured by the weight sensor 12 is referred to as W, the volume of the urine contained in the vessel 10 is referred to as v, the sectional area of the vessel 10 is referred to as A, the density of the urine is referred to as ρ, and the height of the urine accumulated in the vessel 10 is referred to as h, Equation 1 (W=ρgAh) is established. Further, since a multiplication of the sectional area A of the vessel 10 and the height h of the urine is equal to the volume v of the urine, Equation 1 is expressed as Equation 2 (W=ρgv).

In Equation 2, the density ρ of the urine is regarded as 1 because the density ρ is nearly identical to that of the water, and g is constant because it is a gravity constant. Accordingly, Equation 2 can be expressed as Equation 3 (W=ρgAh=ρgv∝v).

As described above, the change in the weight of the urine is measured during the urination, so that variation of the volume of the urine can be recognized. However, since living body variables actually calculated in order to determine the disease in the prostate correspond to urinary flow rate signals, the variation of the volume of the urine is converted to the urinary flow rate signals. That is, since the urinary flow rate is defined by variation of volume per unit time, the urinary flow rate signals can be calculated by differentiating weight signals proportional to volume as a function of time.

FIG. 2 is a graph illustrating an example in which the uro-flowmetry inspection is performed by the conventional method as described above.

FIG. 2 shows results of the conventional urinary flow rate inspection using volume signals obtained by measuring the weight of urine of a normal person, urinary flow rate signals obtained by differentiating the volume signals, and all diagnosis parameters obtained by analyzing the urinary flow rate signals (difficulty in urination and urinary incontinence, Korean Continence Society, ilchokak, P.331, 2003).

According to the conventional method and apparatus for measuring the urinary flow rate as described above, urine is contained in the vessel, variation of weight of the urine is continuously measured using the weight sensor, and then the volume signals based on the measured variation and the urinary flow rate signals obtained by differentiating the volume signals are inspected.

However, in a case where the urine 13 is accumulated in the vessel 10 during the urination, when the urine stream (dotted line in FIG. 1) directly reaches the bottom surface of the vessel 10, additional impact is applied to the bottom surface of the vessel 10 by the momentum (mass×velocity) of the urine stream in addition to the weight of the urine. Thus, the impulse is given to the weight sensor 12 together with the weight of the urine, and the impact effect is irregularly given to the weight sensor 12 according to the amount and speed of the urine stream, so that measurement noise may occur, deteriorating the reliability in the differentiation process for obtaining the urinary flow rate and the process for acquiring_diagnosis parameters.

FIG. 3 is a graph illustrating experimental results using the conventional method as described above. That is, FIG. 3 is a graph illustrating results of experiment in which water of 800 mL, instead of urine, is poured into the vessel similarly to the urination state, and volume signals are obtained by measuring the weight of the water. From the result of the experiment, it can be understood that measurement noise exists in all signals. In order to prevent such an impact effect, an additional funnel is typically used in order to allow urine to flow down along the wall surface of the vessel and to minimize the impulse. However, the funnel must be precisely designed matching with a collection vessel and must be fitted in the collection vessel in use, causing inconvenience to the user.

Further, since the load-cell, which is a conventional sensor device used for weight measurement, is very expensive (at least 70,000 won in Korea), and a high-quality product having the high measurement accuracy is also expensive (more than one million won), the manufacturing cost of a measurement apparatus significantly increases, resulting in economic loss.

### SUMMARY OF THE INVENTION

The present invention has been made to solve the above problems occurring in the prior art, and an object of the present invention is to provide a method and an apparatus for measuring a urinary flow rate using a manometer, in which volume signals are obtained by measuring water pressure using a general-purpose pressure sensor with a low price instead of measuring weight using an expensive load-cell, thereby preventing impact noise and acquiring economic gain with no use of an additional funnel.

In order to accomplish the above object, the present invention provides a method including the steps of: transferring water pressure of urine accumulated in a vessel to an external pressure sensor through a pressure transfer pipe; converting the received water pressure into electrical signals through the pressure sensor, in which the electrical signals are voltage signals corresponding to volume signals; amplifying the electrical signals through a bridge amplifier; converting the amplified analog signals into digital signals (water pressure signals); converting the water pressure signals into volume signals through an interface with an analyzer; and acquiring urinary flow rate signals by differentiating the volume signals. The analyzer can use a computer or a dedicated analyzer.

Since the pressure sensor is the Wheatstone bridge sensor including four resistors, it is possible to use the “multi-purpose bridge amplifier” disclosed in Korea Utility Model Registration No. 404559 issued to present applicant, or a dedicated bridge amplification circuit can be used. An RS-232C serial communication port or a device interfacable with the analyzer is used as an interface with the analyzer.

In order to accomplish the above object, the present invention provides a system including: a vessel in which urine is accumulated; a pressure transfer pipe inserted inside the vessel and connected to an external pressure sensor; an amplifier for extracting and amplifying pressure signals; a converter for converting the amplified pressure signals into digital signals; and an analyzer interfaced with the converter.

The analyzer can use a dedicated analyzer or a computer, and the interface can use an RS-232C serial communication port.

### BRIEF DESCRIPTION OF THE DRAWINGS

The above and other advantages of the present invention will become readily apparent with reference to the following detailed description when considered in conjunction with the accompanying drawings wherein:

FIG. 1 is a conceptual view of a conventional uro-flowmetry scheme;

FIG. 2 is a graph illustrating results of uro-flowmetry inspection of a normal person according to the prior art;

FIG. 3 is a graph illustrating experimental results according to the prior art;

FIG. 4 is a conceptual view of an uro-flowmetry method according to the present invention;

FIG. 5 is a view schematically illustrating a method according to the present invention; and

FIG. 6 is a graph illustrating experimental results according to the present invention;

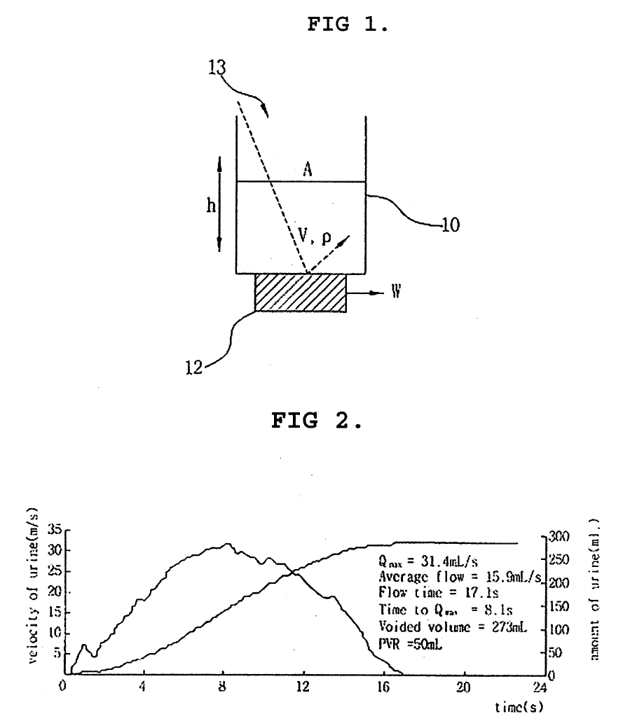

### DESCRIPTION OF THE EMBODIMENTS

Hereinafter, a preferred embodiment of the present invention will be explained in detail with reference to the accompanying drawings.

According to the present invention, as shown in FIG. 4, as urine is accumulated in a vessel 10 having a constant sectional area A, water pressure P in the bottom surface of the vessel 10 is proportional to the height h of the accumulated urine 113, so Equation 4 (P=ρgh) is established.

Equation 4 can be expressed as Equation 5

$$( P = \frac{ρ g A h}{A} )$$

by multiplying the denominator and numerator of Equation 4 by the sectional area A.

In Equation 5, since the volume V of the urine 113 is obtained by multiplying the height h of the urine 113 by the sectional area A of the vessel 10, Equation 5 can be expressed as Equation 6

$$( P = \frac{ρ g}{A} \cdot V ) .$$

In Equation 6, the density ρ of the urine 113 is regarded as 1 because the density ρ is nearly identical to the density of the water, and g is constant because it is a gravity constant. Accordingly, Equation 6 can be expressed as Equation 7

$$( P = \frac{ρ g}{A} \cdot V ∝ V ) .$$

In Equation 7, the pressure P is proportional to the volume V.

Accordingly, once the sectional area A of the vessel 10 is constant, the water pressure P in the bottom surface of the vessel 10 is proportional to the volume V of the accumulated urine 113, so that urinary flow rate signals can be obtained in a method simpler than the conventional method by measuring the water pressure P instead of the weight w, and thus the uro-flowmetry inspection can be performed.

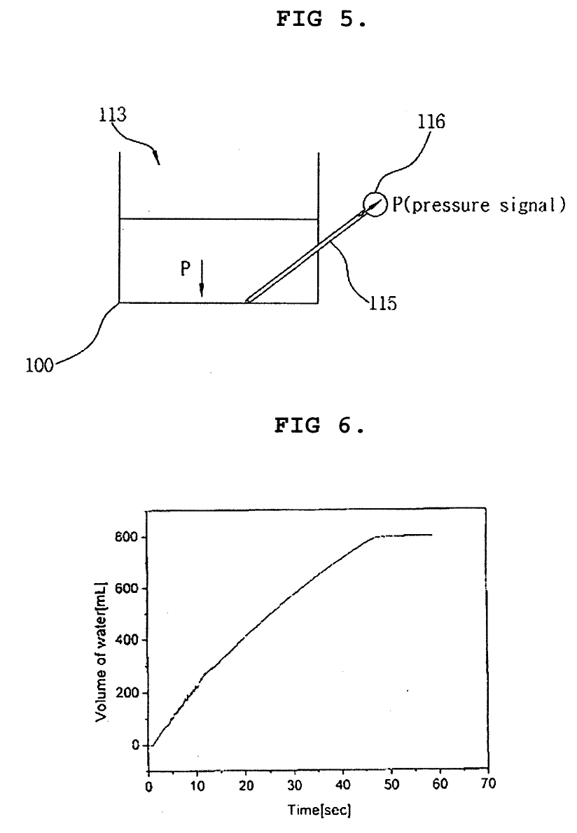

That is, as shown in FIG. 5, as an elongated pressure transfer pipe 115 is installed at the bottom surface of the vessel 10, the pressure P based on the amount of the urine 113 is transferred to an external pressure sensor 116 through the pressure transfer pipe 115 according to the Pascal's principle representing that the pressure P in the bottom surface is uniformly transferred in all directions. The pressure sensor 116 converts water pressure into voltage, thereby obtaining voltage signals corresponding to volume signals.

According to the present invention in which the water pressure P is measured instead of the weight w, if small quantity of the urine 113 is accumulated to the extent that the inlet of the pressure transfer pipe 115 is immersed in the urine 113, only pressure proportional to the height h of the accumulated urine 113 is transferred to the pressure sensor 116 and the impulse to the bottom surface of the vessel 10 is not transferred to the pressure transfer pipe 115, so that the impact noise occurring in the prior art does not occur.

Since the pressure sensor 116 is the Wheatstone bridge sensor including four resistors, a conventional pressure sensor can be used. Specifically, it is possible to use the “multi-purpose bridge amplifier” disclosed in Korea Utility Model Registration No. 404559 issued to present applicant, or a dedicated bridge amplification circuit can be used to extract and amplify pressure signals. In this case, the amplified pressure signals are subject to A/D conversion to make an interface with a computer PC.

An RS-232C serial communication port is used to make the interface with the computer PC, and the computer converts water pressure signals into volume signals, differentiates the volume signals to obtain urinary flow rate signals, and then acquires all diagnosis parameters.

All circuits and analysis techniques applied to the signal accumulation correspond to widely-used general-purpose technology.

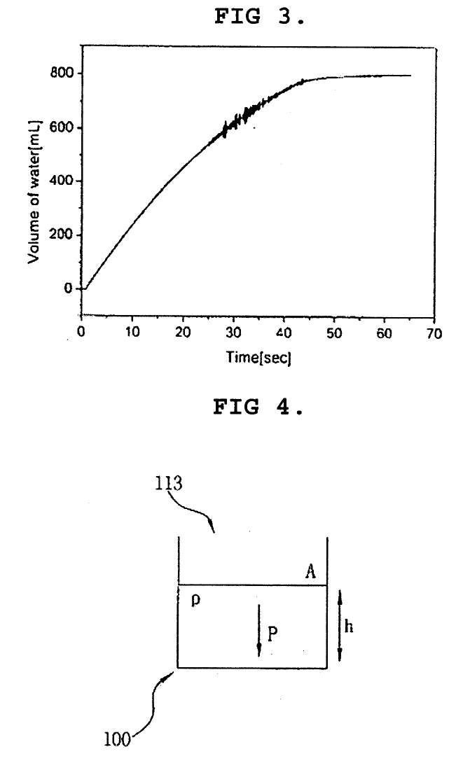

FIG. 6 is a graph illustrating experimental results according to the method of the present invention as described above. That is, FIG. 6 is a graph illustrating results of experiment in which water of 800 mL is poured into the vessel similarly to the urination state and the water pressure is measured under the same condition as that of FIG. 3. In other words, the pressure P is measured instead of the weight W to obtain the volume signals.

As it can be seen from FIG. 6, as compared with the volume signals obtained through weight measurement (see FIG. 3), the volume signals obtained through pressure measurement according to the present invention are very clean signals with nearly no noise. This means that the uro-flowmetry inspection can be performed with high reliability when adopting the water pressure measurement method, instead of the weight measurement method, because the water pressure measurement method can provide high-quality signals while removing impact noise.

Further, the load-cell used for obtaining the graph in FIG. 3 uses the BCL-2L. (CAS Corp., U.S.A) having the lowest price (70,000 won), but the pressure sensor used for obtaining the graph in FIG. 6 uses MPX-1ODP (Freescale, U.S.A) purchasable for the low price of 10,000 won. As a result, the method and the system according to the present invention are more economic than the prior art.

According to the present invention as described above, uro-flowmetry inspection with no measurement noise can be performed using a low-priced pressure sensor even without using the conventional expensive weight sensor. Further, an additional funnel used in order to prevent impact noise in the conventional weight measurement is not employed, so that it is possible to more economically and efficiently obtain urinary flow rate signals, and thus to provide urinary flow rate measuring method and apparatus that can be efficiently used for determining the disease in the prostatomegaly in the masculine.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

1. A method for measuring a urinary flow rate in diagnosis of prostatomegaly, the method comprising the steps of:
transferring water pressure of urine accumulated in a vessel to an external pressure sensor through a pressure transfer pipe;
converting the received water pressure into electrical signals through the pressure sensor, in which the electrical signals are voltage signals corresponding to volume signals;
amplifying the electrical signals through a bridge amplifier;
converting the amplified analog signals into digital signals (water pressure signals);
converting the water pressure signals into volume signals through an interface with an analyzer; and
acquiring urinary flow rate signals by differentiating the volume signals.

2. The method as claimed in claim 1, wherein the pressure transfer pipe has a diameter of 0.1 to 2 mm and the pressure sensor comprises a Wheatstone bridge sensor including four resistors.

3. The method as claimed in claim 1, wherein the interface with the analyzer is carried out through an RS-232C serial communication port.

4. The method as claimed in claim 1, wherein the analyzer comprises a computer or a dedicated analyzer.

5. A system for performing the method as claimed in claim 1, the system comprising:
a vessel in which urine is accumulated;
a pressure transfer pipe inserted inside the vessel and connected to an external pressure sensor;
an amplifier for extracting and amplifying pressure signals;
a converter for converting the amplified pressure signals into digital signals; and
an analyzer interfaced with the converter.

6. The system as claimed in claim 5, wherein the pressure transfer pipe has a diameter of 0.5 to 2 mm and the pressure sensor comprises a bridge amplification circuit including four resistors.

7. The system as claimed in claim 5, wherein the interface is carried out through an RS-232C serial communication port.

8. The system as claimed in claim 5, wherein the analyzer comprises a computer or a dedicated analyzer.

9. A system for performing the method as claimed in claim 2, the system comprising:
a vessel in which urine is accumulated;
a pressure transfer pipe inserted inside the vessel and connected to an external pressure sensor;
an amplifier for extracting and amplifying pressure signals;
a converter for converting the amplified pressure signals into digital signals; and
an analyzer interfaced with the converter.

10. A system for performing the method as claimed in claim 3, the system comprising:
a vessel in which urine is accumulated;
a pressure transfer pipe inserted inside the vessel and connected to an external pressure sensor;
an amplifier for extracting and amplifying pressure signals;
a converter for converting the amplified pressure signals into digital signals; and
an analyzer interfaced with the converter.

11. A system for performing the method as claimed in claim 4, the system comprising:
a vessel in which urine is accumulated;
a pressure transfer pipe inserted inside the vessel and connected to an external pressure sensor;
an amplifier for extracting and amplifying pressure signals;
a converter for converting the amplified pressure signals into digital signals; and
an analyzer interfaced with the converter.
